(19) United States
(12) Patent Application Publication
(10) Pub. No.: US 2022/0098638 A1
(43) Pub. Date: Mar. 31, 2022

(54) MULTIPLEX PCR METHOD USING APTAMER

(71) Applicant: APTAMER SCIENCES INC., Gyeonggi-do (KR)

(72) Inventors: Kiseok Kim, Gyeonggi-do (KR); Euisu Shin, Gyeongsangbuk-do (KR); Juhyung Kang, Gyeonggi-do (KR); Minyoung Yeo, Gyeonggi-do (KR); Kwang-hyun Lee, Gyeonggi-do (KR)

(73) Assignee: APTAMER SCIENCES INC., Gyeonggi-do (KR)

(21) Appl. No.: 17/427,266

(22) PCT Filed: Dec. 9, 2019
(86) PCT No.: PCT/KR2019/017271

§ 371 Date: Jul. 30, 2021

(30) Foreign Application Priority Data
Feb. 1, 2019 (KR) 10-2019-0013775

(51) Int. Cl. C12Q 1/6804; C12Q 1/686; G01N 33/53

(52) U.S. Cl. CPC C12Q 1/6804; G01N 33/5308; C12Q 1/686

(57) ABSTRACT

Provided are a method of detecting a target molecule and a composition for detecting a target molecule, the composition including an aptamer that recognizes a target region of a target molecule and the method using the aptamer as a template for a polymerase chain reaction (PCR).

### TECHNICAL FIELD

The present invention relates to a method of detecting a target molecule and a composition for detecting a target molecule, the composition including an aptamer that recognizes a target region of a target molecule and the method using the aptamer as a template for a polymerase chain reaction (PCR).

### BACKGROUND ART

Aptamer is single-stranded nucleic acid (DNA, RNA, or modified nucleic acid) that binds to a specific target with high affinity and specificity. Aptamers are generally obtained via systematic evolution of ligands by exponential enrichment (SELEX). Aptamers discovered in this way are used for diagnostic or therapeutic purposes because they specifically bind to various target molecules ranging from small target chemical molecules, such as antibodies, to proteins with a picomolar level dissociation constant. Aptamers are easier to produce than antibodies and have higher stability than antibodies since they can be stored at room temperature. Due to such superior effects of aptamers compared to antibodies, extensive research has been conducted into aptamers used as biomarkers and biosensors for diagnosis thereof.

Enzyme-linked immunosorbent assay (ELISA) is as an immunodiagnostic method related to detection and quantification of physiologically important molecules. ELISA includes 1) a method of identifying a target by coating an antigen on a plate, binding an antibody thereto, and analyzing a signal therefrom and 2) a sandwich method of identifying a target by coating an antibody on a plate, treating the plate with an antigen, and then treating the plate with another antibody. Among these, the sandwich method is mainly used and for detection and quantification of a protein, and when a target biomarker protein binds to an antibody immobilized on the surface and another antibody binds thereto as a target, the amount of a target substance may be measured. Because the assay uses antibodies and enzymes, 1) problems caused by reproducibility and batch-to-batch variation may occur, 2) it is significantly affected by temperature during distribution and storage, 3) it is difficult to simultaneously detect a plurality of biomarkers since one biomarker is detectable in a single well, and 4) it is difficult to detect a small amount of an antigen since detection is performed using an enzyme.

As a representative method of molecular diagnosis, polymerase chain reaction is a method of amplifying a tiny amount of a particular DNA into a large amount within a short period of time and may increase sensitivity using a small amount of sample. In addition, detection and quantification of a sample are possible via real-time polymerase chain reaction. Immuno-PCR, as a combination of these diagnostic methods, is a diagnostic method capable of detecting a small amount of protein by binding DNA to an end of an antibody used in ELISA and amplifying the DNA and has a sensitivity 100 to 10000 times higher than that of general ELISA. However, Immuno-PCR has the same problems as ELISA described above since antibodies are used therein, and also, it is difficult to immobilize oligo-DNA to an antibody and commercialize this method due to low yield.

### DISCLOSURE

### Technical Problem

As a result of intensive efforts to overcome all disadvantages of antibody-based techniques and to develop techniques capable of detecting and quantifying one or more target molecules, the present inventors have found a method of detecting a target molecule using a nucleic acid aptamer as a template for a polymerase chain reaction (PCR) and confirmed that one or more target molecules may be detected and quantified using the method, thereby completing the present invention.

### Technical Solution

An object of the present invention is to provide a method of detecting a target molecule by using an aptamer, which recognizes a target region of the target molecule, as a template for a polymerase chain reaction (PCR).

Another object of the present invention is to provide a composition for detecting a target molecule including an aptamer recognizing a target region of the target molecule wherein the aptamer is used as a template for a polymerase chain reaction (PCR).

### Advantageous Effects

The aptamer of the present invention has excellent binding affinity to a specific biomarker, and thus the biomarker may be diagnosed and quantified by isolating the aptamer bound to the biomarker and performing real-time polymerase chain reaction using primers for the aptamer. In addition, by diagnosing and quantifying three or more biomarkers using three or more aptamers and primers therefor, various types of biomarkers may be simultaneously diagnosed.

### BRIEF DESCRIPTION OF DRAWINGS

FIG. 1 is a schematic diagram of an aptamer-based multiplex polymerase chain reaction.

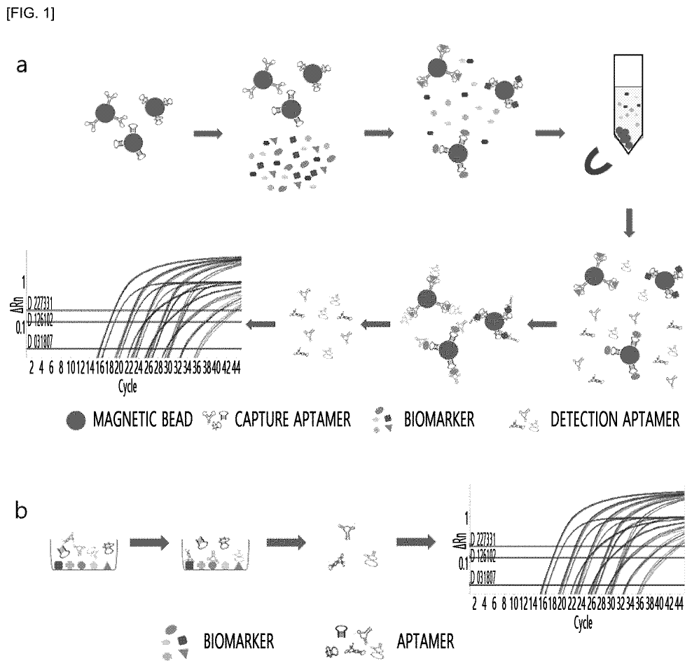

FIG. 2 shows results of finding concentration conditions of bovine serum albumin (BSA) suitable for minimizing a detection aptamer non-specifically binding to magnetic beads.

FIG. 3 shows results of finding concentration conditions of dextran sulfate (DₓSO₄) suitable for minimizing a detection aptamer non-specifically binding to magnetic beads.

FIG. 4 shows results of amplification curves of aptamer pairs in real-time PCR for target and non-target biomarkers in single diagnosis. Specifically, it is confirmed that the aptamers bind to the target biomarkers in the presence of the target biomarkers so that the curves appear early.

FIG. 5 shows amplification curves of real-time PCR in single diagnosis using aptamer pairs for target biomarkers compared with amplification curves thereof in multiple diagnosis. Specifically, it is confirmed that amplification curves of the target aptamers of single diagnosis are the same as those of multiple diagnosis in the presence of the same biomarker.

FIG. 6 shows amplification curves of PCR for testing performance of aptamers for target biomarkers in multiple diagnosis using aptamer pairs. Specifically, it is confirmed that the target biomarkers are detectable even with decreased amounts.

FIG. 7 shows results of amplification curves of single aptamers in real-time PCR for target and non-target biomarkers in single diagnosis. Specifically, it is confirmed that the aptamers bind to the target biomarkers in the presence of the target biomarkers so that the curves appear early.

FIG. 8 shows amplification curves of real-time PCR in single diagnosis using single aptamers for target biomarkers compared with amplification curves thereof in multiple diagnosis. Specifically, it is confirmed that amplification curves of the target aptamers of single diagnosis are the same as those of multiple diagnosis in the presence of the same biomarker.

FIG. 9 shows amplification curves of PCR for testing performance of aptamers for target biomarkers in multiple diagnosis using single aptamers. Specifically, it is confirmed that the target biomarkers are detectable even with decreased amounts.

### BEST MODE

An aspect of the present invention to achieve the above-described objects provides a method of detecting a target molecule by using an aptamer, which recognizes a target region of the target molecule, as a template for a polymerase chain reaction (PCR).

In most cases, enzyme-linked immunosorbent assay (ELISA) or immuno-polymerase chain reaction (Immuno-PCR) are generally used to detect and quantify a target molecule. In the case of ELISA or Immuno-PCR, there may be problems in terms of reproducibility, stability, difficulty in simultaneous detection of a plurality of biomarkers, and the like because antibodies and enzymes are used. It is difficult to commercialize ELISA or Immuno-PCR due to difficulty in immobilization of oligo-DNA on an antibody and low yield. However, by using the method of detecting a target molecule using an aptamer recognizing a target region of the target molecule according to the present invention, the aptamer bound to the target molecule may be used as a template for PCR, and a plurality of target molecules may be detected simultaneously using a number of separate aptamers.

Specifically, the method of the present invention includes: (i) bringing an aptamer recognizing a target region of a target molecule into contact with the target molecule; and (ii) performing a polymerase chain reaction (PCR) using, as a template, an aptamer forming a complex via the contact and a bound aptamer in a complex of the target molecule, but is not limited thereto.

Throughout the specification, the term “target molecule” refers to a substance detectable by the aptamer of the present invention. Specifically, the target molecule may be present in an isolated sample and may include at least one selected from the group consisting of a protein, a peptide, a carbohydrate, a polysaccharide, a glycoprotein, a hormone, a receptor, an antigen, an antibody, a virus, a cofactor, a drug, a dye, a growth factor, and a controlled substance to which a capture aptamer binds, without being limited thereto. In view of the objects of the present invention, the target molecule or the target region may be one or more types and is not particularly limited as long as the target molecule or the target region is recognizable by an aptamer.

In addition, as a target substance to which an aptamer binds with high affinity and specificity in view of the objects of the present invention, the type of the target molecule is not particularly as long as the target molecule is a protein capable of binding to a first capture aptamer or a first capture aptamer in view of the objects of the present invention. Specifically, at least one selected from the group consisting of animal cell membrane protein, plant cell membrane protein, microorganism cell membrane protein, and virus protein may be used without being limited thereto. According to an embodiment of the present invention, IR, ErbB2, and VEGFR2 were used as target molecules.

Also, the term “sample” may include at least one selected from the group consisting of a biological sample, an environmental sample, a chemical sample, a pharmaceutical sample, a food sample, an agricultural sample, and a livestock sample. Specifically, the sample may include at least one selected from the group consisting of whole blood, leukocytes, peripheral blood mononuclear cells, plasma, serum, sputum, breath, urine, semen, saliva, meningeal fluid, amniotic fluid, glandular fluid, lymph fluid, nipple aspirate, bronchial aspirate, synovial fluid, joint aspirate, cells, cell extract, stool, tissue extract, biopsy tissue, and cerebrospinal fluid, without being limited thereto.

As used herein, the term “aptamer” refers to a particular type of single-stranded nucleic acid, double-stranded nucleic acid, or peptide having a stable three-dimensional structure and binding to a target molecule with high affinity and specificity. Specifically, the aptamer may be DNA, RNA, or any combination thereof, but is not limited thereto. In addition, the aptamer may be a non-modified, i.e., natural, aptamer or a modified aptamer. Specifically, the modified aptamer may include at least one chemical modification, and the at least one chemical modification refers to chemical substitution at one or more positions independently selected from positions of a ribose, a deoxyribose, a phosphate, and a base. In addition, the chemical modification may be selected from the group consisting of 2′-position sugar modification, purine modification at 2′-fluoro (2′-F), 2′-O-methyl, and 8-positions, modification on exocyclic amines of cytosine, substitution of 5-bromouracil, substitution of 5-bromodeoxyuridine, substitution of 5′-bromodeoxycitidine, modification of backbone, methylation, 3′ cap, and 5′ cap, without being limited thereto. The base used other than the modified base may be selected from the group consisting of bases A, G, C, and T and deoxy forms (e.g., 2′-deoxy forms) thereof, unless otherwise stated. The modified base refers to a modified form by substitution of the 5-position of deoxyuracil (dU) with a hydrophobic functional group and may be used to replace the base “T”. In addition, the hydrophobic functional group may include at least one selected from the group consisting of a benzyl group, a naphthyl group, a pyrrolebenzyl group, and tryptophan. As described above, modification occurs by substitution of the 5-position of the dU base with a hydrophobic functional group and thus the modified form has significantly improved affinity to periostin compared to non-modified form.

In an embodiment of the present invention, non-modified aptamers and modified aptamers are shown in Table 1.

The aptamer of the step (i) may be an aptamer pair including a capture aptamer recognizing a target region and a detection aptamer recognizing a target region and used as a template, or a single aptamer recognizing a target region and used as a template, but is not limited thereto. The aptamer pair or the single aptamer may be present in the same number of types as that of the target molecule or target region corresponding to the types of target molecules or target regions. For example, the aptamer pair or single aptamer may be one aptamer pair or single aptamer having high affinity and specificity to one target molecule or target region or one or more aptamer pairs or single aptamers binding to one or more target molecules or target regions. As another example, when there is one type of target molecule, one type of aptamer may be used, when there are two types of target molecules, two types of aptamers may be used, and when there is three types of target molecules, three types of aptamers may be used, and thus the number of types of aptamer is determined in accordance with types of the target molecules. In this regard, because there may be a plurality of target regions in a target molecule, more types of aptamers than those of target molecules may be used.

By using the aptamer pair, a target molecule floating in a sample may be detected, and a target molecule may be immobilized on a support in the case of the single aptamer, without being limited thereto.

In the present invention, the aptamer pair and the single aptamer as described above may be used, and a specific method related thereto is as follows.

In view of the objects of the present invention, the method of using the aptamer pair may include the following steps. Specifically, the method may include: (a) forming a first complex by binding a first capture aptamer to a solid support; (b) binding a target molecule to the first complex of the step (a); (c) forming a second complex by binding a second capture aptamer to the target molecule of the step (b); and (d) isolating the second capture aptamer from the second complex of the step (c) and performing a polymerase chain reaction (PCR), without being limited thereto.

Individual steps of the method of detecting a target molecule using the aptamer pair will be described in detail.

In the step (a), the first capture aptamer is bound to the solid support to form the first complex.

As used herein, the term “first capture aptamer” refers to an aptamer capable of recognizing a target region of a target molecule present in an isolated sample after binding to a solid support. In view of the objects of the present invention, the first capture aptamer may be an aptamer conjugated with a marker at the 5′-end thereof, and this term may be used interchangeably with grab aptamer.

The first capture aptamer may be labeled with a detectable molecule such as a radioisotope, a fluorescent compound, a bioluminescent compound, a chemical luminescent compound, a metal chelate, or an enzyme. Specifically, the labeling may be performing by inserting a marker detectable by one method selected from the group consisting of spectroscopic, photochemical, fluorescent, biochemical, immunochemical, and chemical methods. As effective detection molecules, a radioactive substance (³²P, ³⁵S, ³H, and ¹²⁵I), a fluorescent dye (5-bromodeoxyuridine, fluorescein, acetylaminofluorene, or digoxigenin), biotin, or the like may be used. For example, the first capture aptamer according to the present invention may be one conjugated with biotin at the 5′-end thereof, without being limited thereto.

The method may further include selectively removing the first capture aptamer not bound to the first complex, but is not limited thereto. For example, in an embodiment of the present invention, the resultant was washed with Washing Buffer 1 to remove the capture aptamer not bound to the first complex.

The “solid support” includes at least one selected from the group consisting of a magnetic bead, a polymer bead, an agarose bead, a polystyrene bead, an acrylamide bead, a solid core bead, a porous bead, a paramagnetic bead, a glass bead, a controlled pore bead, a microtiter well, a cycloolefin copolymer substrate, a membrane, a plastic substrate, nylon, a Langmuir-Blodgett film, glass, a germanium substrate, a silicon substrate, a silicon wafer chip, a flow through chip, a microbead, a polytetrafluoroethylene substrate, a polystyrene substrate, a gallium arsenide substrate, a gold substrate, and a silver substrate, and may specifically be a magnetic bead, without being limited thereto.

The method may further include blocking the solid support using a blocking buffer to prevent non-specific binding thereof before binding the aptamer to the solid support in the step (a), but is not limited thereto.

Specifically, the blocking buffer may include at least one selected from the group consisting of bovine serum albumin (BSA), salmon sperm DNA, herring sperm DNA, skim milk, and casein, and may be more specifically BSA, without being limited thereto.

The step (b) is a step of binding the target molecule to the first complex of the step (a).

In view of the objects of the present invention, this is a step of binding the target molecule to the first complex prepared by binding the first capture aptamer to the solid support, and the target molecule refers to a target substance to which the aptamer binds with high affinity and specificity as described above.

The method may further include selectively removing the target molecule not bound to the first complex, but is not limited thereto. For example, in an embodiment of the present invention, the resultant was washed with Washing Buffer 1 to remove the target molecule not bound to the first complex.

The step (c) is a step of forming a second complex by binding a second capture aptamer to the target molecule of the step (b).

As used herein, the term “second capture aptamer” refers to a detection aptamer recognizing the target region of the first complex and used as a template. In view of the objects of the present invention, the second capture aptamer may be used interchangeably with the detection aptamer.

The method may further include selectively removing the second capture aptamer not bound to the target region of the first complex, but is not limited thereto. For example, in an embodiment of the present invention, the second complex was washed with Washing Buffer 1 to remove the detection aptamer not bound to the target region of the first complex.

In addition, in view of the objects of the present invention, the method is characterized in that one or more types of target molecule are detected using an aptamer recognizing one or more types of particular antigens including the complex in which the target molecule is bound to the first complex before the step (c), without being limited thereto.

The step (d) is a step of isolating the second capture aptamer from the second complex of the step (c) and performing a polymerase chain reaction (PCR).

As used herein, the term “polymerase chain reaction (PCR)” refers to a process of amplifying a target nucleic acid by repeating denaturation, annealing, and extension using the target nucleic acid as a template and primers specific to the target nucleic acid. In view of the objects of the present invention, the second capture aptamer may be isolated from the second complex, and PCR may be performed using primers specific to the aptamer, without being limited thereto. For example, the PCR may be real-time PCR or multiplex-PCR, and the process of denaturation, annealing, and extension may be repeated once or more due to characteristics of the PCR.

As used herein, the term “multiplex-PCR” indicates that a plurality of target molecules contained in a sample may be amplified simultaneously. In the present invention, one or more types of target molecules are detected using the aptamer, without being limited thereto. The multiplex-PCR may be performed by: (i) simultaneously detecting and quantifying one or more target molecules by simultaneously reacting the target molecules with an aptamer in a well or tube; (ii) simultaneously detecting and quantifying one or more target molecules by reacting the target molecules with an aptamer in one or more wells or tubes, without being limited thereto. Also, in view of the objects of the present invention, the PCR may be real-time PCR, without being limited thereto.

In view of the objects of the present invention, the term “multiplex-PCR” may be used interchangeably with quantitative PCR (qPCR) and real-time PCR. Specifically, the quantitative PCR (qPCR) or real-time PCR are used to detect and quantify nucleic acids in various applications.

The qPCR amplifies nucleic acid via three steps of denaturation, annealing, and extension like standard PCR and enables quantification thereof by collecting data via fluorescent labeling. Specifically, in the case of dye-based qPCR, the fluorescent labeling is performed by using a dsDNA-binding dye, and in the case of probe-based qPCR, target-specific probes need to be optimized and designed as well as primers to simultaneously detect a plurality of target molecules in each sample.

In view of the objects of the present invention, the real-time PCR is characterized in that a TaqMan probe PCR and an intercalating fluorescent dye are used to detect and quantify the target molecule, without being limited thereto.

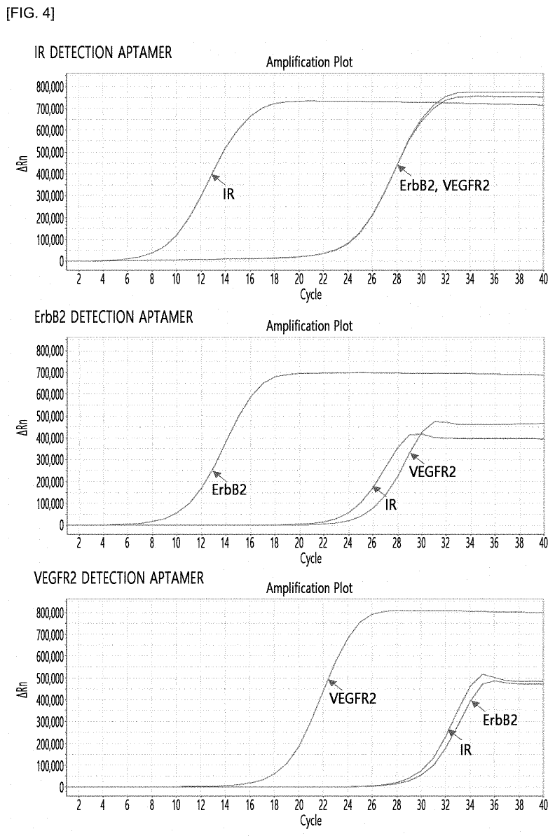

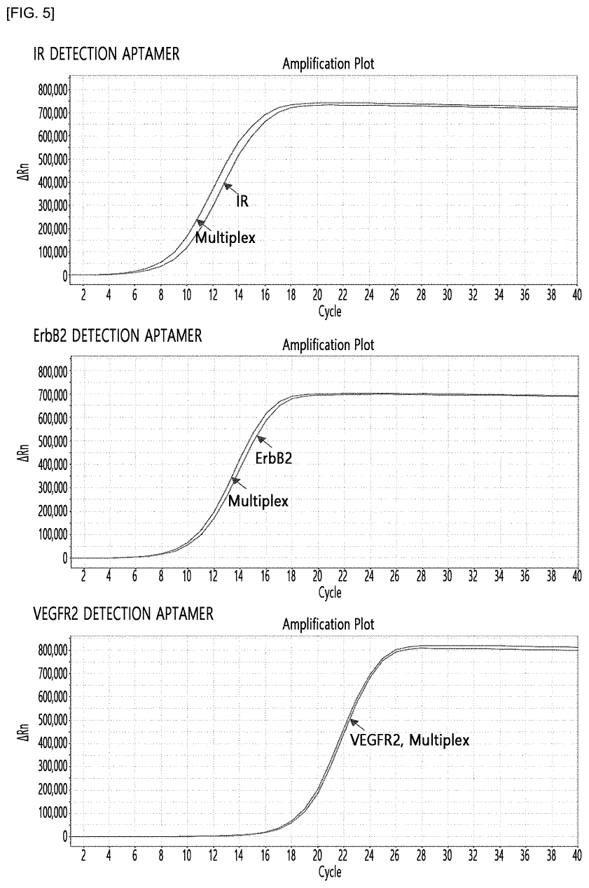

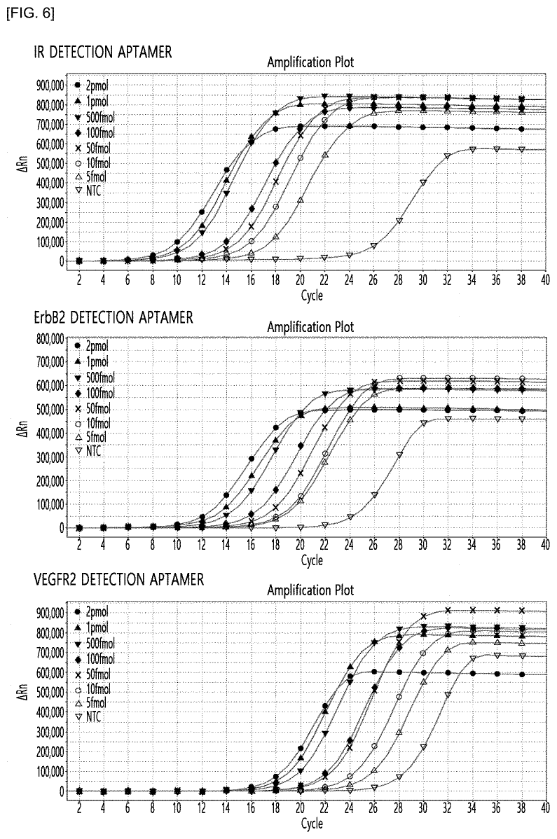

In an embodiment of the present invention, as a result of performing single diagnosis using the aptamer pair, it was confirmed that a curve of the target molecule was observed at a front portion, and it was also confirmed that a curve according to a multiple diagnosis was consistent with the result of the single diagnosis (FIGS. 4 and 5). In addition, as a result of confirming performance of a multiple diagnosis system, it was confirmed that the effects of the multiple diagnosis were obtained in each target molecule (FIG. 6).

Based on these results, it was confirmed that the method of the present invention enables single diagnosis or multiple diagnosis for simultaneously detecting target molecules using the aptamer pair, unlike conventional techniques using antibodies.

In view of the objects of the present invention, the method of using the single aptamer may include the following steps. Specifically, the method may include: (a) binding a target molecule to a solid support; (b) forming a complex by binding a capture aptamer to the target molecule of the step (a); and (c) isolating the capture aptamer from the complex of the step (b) and performing a polymerase chain reaction (PCR), without being limited thereto.

Some terms and some processes of each step of the method of detecting a target molecule using the single aptamer are the same as those of the method of detecting a target molecule using the aptamer pair.

The method, unlike the method using the aptamer pair, does not include the step of forming of the first complex by binding the first capture aptamer to the solid support, but includes binding the target molecule to the solid support, but is not limited thereto.

As used herein, the term “capture aptamer” refers to a single aptamer that recognizes the target region of the target molecule bound to the solid support in the complex and is used as a template. In view of the objects of the present invention, the capture aptamer may be used interchangeably with the single aptamer.

The method may further include selectively removing the capture aptamer not bound to the target region of the target molecule bound to the solid support in the complex, but is not limited thereto. For example, in an embodiment of the present invention, the complex was washed with Washing Buffer 1 to remove the single aptamer not bound to the target region of the complex.

As used herein, the term “bound complex” may refer to (a) the first complex in which the first capture aptamer is bound to the solid support, (b) the complex in which the target molecule is bound to the first complex, (c) the second complex in which the second capture aptamer is bound to the target molecule, (d) the complex in which the target molecule is bound to the solid support, or (e) the complex in which the capture aptamer is bound to the target molecule, but is not limited thereto.

The method may further include isolating the capture aptamer from the complex and amplifying the isolated capture aptamer, and any method for PCR commonly used in the art may also be added thereto without limitation.

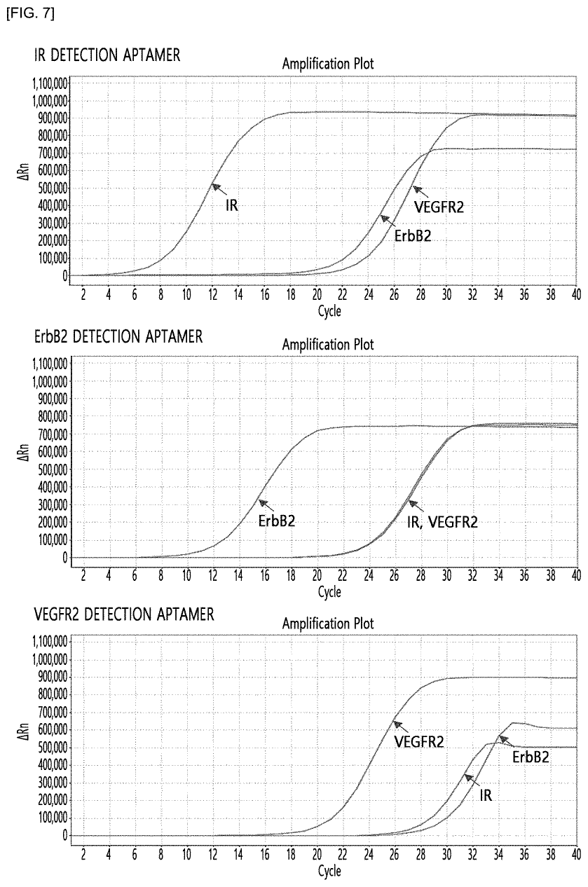

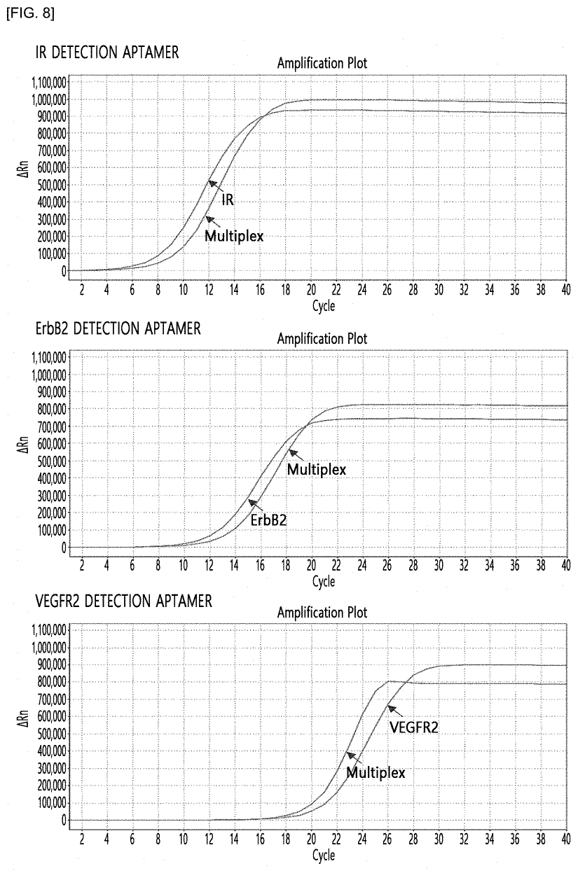

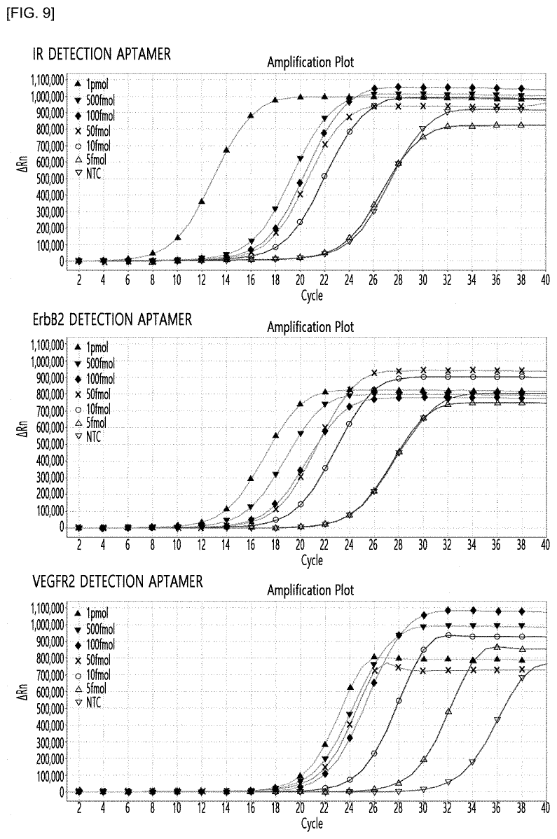

In an embodiment of the present invention, as a result of performing single diagnosis using the single aptamer, it was confirmed that a curve of the target molecule is observed at a front portion, and it was also confirmed that a curve according to a multiple diagnosis was consistent with the result of the single diagnosis (FIGS. 7 and 8). In addition, as a result of identifying performance of a multiple diagnosis system, it was confirmed that the effects of the multiple diagnosis were obtained in each target molecule (FIG. 9).

Based on these results, it was confirmed that the method of the present invention enables single diagnosis or multiple diagnosis for simultaneously detecting target molecules using the single aptamer, unlike conventional techniques using antibodies.

This indicates that a plurality of (one or more) target molecules in a living body may be detected via one reaction by using the aptamer (aptamer pair or single aptamer) stable at a high temperature and easy to store and distribute as a template for polymerase chain reaction.

This also indicates that a plurality of (one or more) target molecules may be simultaneously or concurrently detected and quantified by selecting sequences of primers from aptamers having different base sequences without base sequence interference.

Another aspect of the present invention to achieve the objects provides a composition for detecting a target molecule including an aptamer recognizing a target region of a target molecule, wherein the aptamer is used as a template for polymerase chain reaction (PCR).

It is characterized in that multiplex-PCR is performed using one or more target molecules, and the aptamer may be an aptamer pair including a capture aptamer recognizing a target region and a detection aptamer recognizing a target region and used as a template, or a single aptamer recognizing a target region and used as a template, but is not limited thereto.

### MODE FOR INVENTION

Hereinafter, the present invention will be described in more detail with reference to the following examples. However, the following examples are merely presented to exemplify the present invention, and the scope of the present invention is not limited thereto.

### Example 1: Sandwich-type Assay Using Aptamer Pair

### Example 1-1. Blocking Streptavidin Magnetic Bead Using BSA

20 μg (2 μL) of magnetic beads coated with streptavidin were added to a 1.5 mL tube. A buffer was removed using a magnetic stand. The magnetic beads were washed three times with 100 μL of an SB17 buffer (1×SB17 (5 mM EDTA, 200 mM HEPES, 510 mM NaCl, 25 mM MgCl₂, 25 mM KCl, pH 7.5), and 0.05% tween20). 100 μL of Blocking Buffer 1 (1×SB17, 0.05% tween20, and 10% BSA) was added thereto in a state where only the magnetic beads remained. Blocking was performed using an Eppendorf ThermoMixer C at 600 rpm at room temperature for 1 hour. After blocking, the blocking buffer was removed by washing three times with 100 μL of the SB17 buffer.

### Example 1-2. Immobilization of Capture Aptamer on Streptavidin Magnetic Bead

The magnetic beads prepared in Example 1-1 above were reacted with 20 pmol of a capture aptamer conjugated with biotin at the 5′-end thereof. The reaction was conducted in 100 μL of Binding Buffer 1 (1×SB17, 0.05% tween20, and 20 μM DₓSO₄) in total at 600 rpm at room temperature for 15 minutes. In order to remove the capture aptamer that was not immobilized, the resultant was washed three times with 100 μL of Washing Buffer 1 (1×SB17, 0.05% tween20, and 20 μM DₓSO₄).

### Example 1-3. Binding of Target Molecule

2 pmol of a target molecule was added to the magnetic beads prepared in Example 1-2, followed by reaction in 100 μL of Binding Buffer 1 in total at 600 rpm at room temperature for 10 minutes. The target molecule not bound to the capture aptamer was washed out three times with 100 μL of Washing Buffer 1.

### Example 1-4. Binding and Eluting of Detection Aptamer

2 pmol of a detection aptamer was added to the magnetic beads prepared in Example 1-3, followed by reaction in 100 μL of Binding Buffer 1 in total at 600 rpm at room temperature for 10 minutes. Unreacted detection aptamer was washed out three times with 100 μL of Washing Buffer 1. 30 μL of 2 mM NaOH was added thereto, followed by reaction at 600 rpm at room temperature for 10 minutes to elute the detection aptamer.

### Example 2: Method of Diagnosing Multiple Biomarkers Using Single Aptamer

### Example 2-1. Immobilization of Protein in Plate Well

In order to immobilize a target molecule, a protein was diluted using a carbonate-bicarbonate solution (pH 9.6). 100 μL of the diluted solution was added to each well of an ELISA-plate, followed by reaction at 4° C. and 600 rpm for 12 hours. Unreacted protein was washed out once with 100 μL of Washing Buffer 1 at room temperature for 2 minutes at 600 rpm.

### Example 2-2. Blocking of Plate Well Using BSA

200 μL of a blocking buffer (3% BSA) was added to the plate, followed by blocking at room temperature for 1 hour at 600 rpm. The resultant was washed twice with 100 μL of Washing Buffer 1 at room temperature for 2 minutes at 600 rpm.

### Example 2-3. Binding and Eluting of Aptamer

The target molecule was added to each well of the plate, followed by reaction at room temperature at 800 rpm for 1 hour. The resultant was washed three times with 100 μL of Washing Buffer 5 for 2 minutes at 600 rpm. 30 μL of 2 mM NaOH was added thereto, followed by reaction at 600 rpm at room temperature for 10 minutes to elute a detection aptamer.

### Example 3: Multiplex Real-time PCR

A real-time PCR mixture solution (1×Taq buffer (SolGent), 0.2 mM dNTP, 0.2 μM primer, 5 mM MgCl₂, 1×SYBR, and 0.05 U Taq polymerase (SolGent)) was prepared. 18 μL of the prepared real-time PCR mixture solution and 2 μL of the eluted detection aptamer were added to a real-time PCR tube. Samples were analyzed using an Applied Biosystems 7500 real-time PCR system. The real-time PCR proceeded by conducting a first process of maintaining at 96° C. for 15 seconds, at 55° C. for 10 seconds, and at 70° C. for 30 minutes once and then repeating a second process of maintaining at 96° C. for 15 seconds and at 70° C. for 1 minute 40 times.

### Experimental Example 1: Removal of Non-Specific Signal of Detection Aptamer by Blocking Test of Magnetic Beads Using Different Concentrations of BSA

In order to remove non-specific signals between magnetic beads and an aptamer, a blocking test was performed using different concentrations of BSA. 20 μg of streptavidin-coated magnetic beads were added to a 1.5 mL tube, and a buffer was removed using a magnetic stand. Specifically, the resultant was washed three times with 100 μL of the SB17 buffer. 100 μL of Blocking Buffer 2 (1×SB17, 0.05% tween20, and 3% or 10% BSA) was added thereto in a state where only the magnetic beads remained. Blocking was performed at 600 rpm at room temperature for 1 hour. The remaining blocking buffer was removed by washing three times with 100 μL of the SB17 buffer.

The magnetic beads prepared as described above were reacted with 20 pmol of a capture aptamer (SEQ ID NO: A1, A3, or A5) conjugated with biotin at the 5′-end thereof. The reaction was conducted in 100 μL of Binding Buffer 1 in total at 600 rpm at room temperature for 15 minutes. In order to remove the capture aptamer that was not immobilized, the resultant was washed three times with 100 μL of Washing Buffer 1.

30 μL of 2 mM NaOH was added to the magnetic beads prepared as described above, followed by reaction at 600 rpm at room temperature for 10 minutes to elute the aptamer bound to the magnetic beads.

Real-time PCR Mixture Solution 1 (1×Taq buffer (SolGent), 0.2 mM dNTP, 0.2 μM primer (P1, P2, P3, P4, P5, or P6), 5 mM MgCl₂, 1×SYBR, and 0.05 U Taq polymerase (SolGent)) was prepared. 18 μL of the prepared real-time PCR mixture solution and 2 μL the eluted detection aptamer were added to a real-time PCR tube. Samples were analyzed using an Applied Biosystems 7500 real-time PCR system. The real-time PCR proceeded by conducting a first process at 96° C. for 10 minutes once and then repeating a second process at 96° C. for 15 seconds and at 60° C. for 1 minute 40 times.

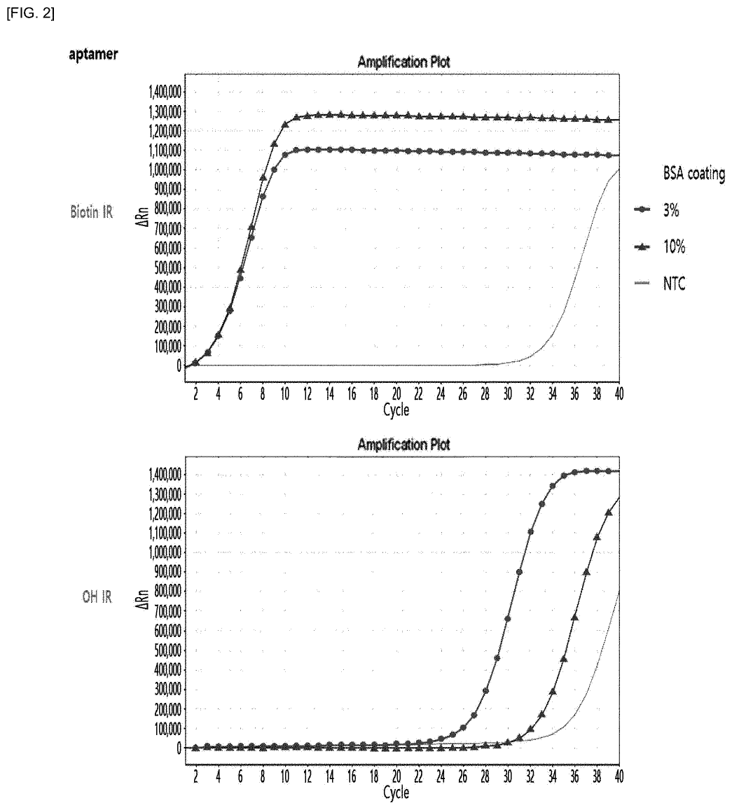

Six samples were prepared using one type of aptamer (IR) as described above, and results as shown in FIG. 2 were obtained.

As a result, as shown in FIG. 2, while the capture aptamer is bound to streptavidin regardless of the blocking buffer, it was confirmed that non-specific binding was almost removed in the case of the detection aptamer according to the concentration of BSA.

### Experimental Example 2: Removal of Non-Specific Signal of Detection Aptamer by Test with Different Concentrations of DₓSO₄

In order to remove non-specific signals, a test was performed using different concentrations of DₓSO₄ used as a competitor in each step.

### Experimental Example 2-1: Test with Different Concentrations of DₓSO₄ in Binding Step

20 μg (2 μL) of streptavidin-coated magnetic beads were added to a 1.5 mL tube. A buffer was removed using a magnetic stand. The resultant was washed three times with 100 μL of the SB17 buffer. 100 μL of Blocking Buffer 1 (1×SB17, 0.05% tween20, and 10% BSA) was added thereto in a state where only the magnetic beads remained. Blocking was performed using an Eppendorf Thermo Mixer C at 600 rpm at room temperature for 1 hour. The remaining blocking buffer was removed by washing three times with 100 μL of the SB17 buffer.

The magnetic beads prepared as described above were reacted with 20 pmol of a capture aptamer (SEQ ID NO: A1, A3, or A5) conjugated with biotin at the 5′-end thereof. The reaction was conducted in 100 μL of Binding Buffer 2 (1×SB17, 0.05% tween20, and 0 μM, 20 μM, 40 μM, or 60 μM DₓSO₄) in total at 600 rpm at room temperature for 15 minutes. In order to remove the capture aptamer that was not immobilized, the resultant was washed three times with 100 μL of Washing Buffer 2 (1×SB17 and 0.05% tween20).

2 pmol of a target molecule or non-target molecule (IR (recombinant human insulin receptor protein, R&D systems, 1544-IR-050), ErbB2 (recombinant human ErbB2 protein, R&D systems, 1129ER-050), or VEGFR2 (human VEGF R2 protein, Acro Biosystems, KDR-H5227)) was added to the magnetic beads prepared as described above, followed by reaction in 100 μL of Binding Buffer 2 in total at 600 rpm at room temperature for 10 minutes. The target molecules not bound to the capture aptamer was washed out three times with 100 μL of Washing Buffer 2.

2 pmol of a detection aptamer (SEQ ID NO: A2, A4, or A6) was added to the magnetic beads prepared as described above, followed by reaction in 100 μL of Binding Buffer 2 in total at 600 rpm at room temperature for 10 minutes. Unreacted detection aptamer was washed out three times with 100 μL of Washing Buffer 2. 30 μL of 2 mM NaOH was added thereto, followed by reaction at 600 rpm at room temperature for 10 minutes to elute the detection aptamer.

Real-time PCR mixture solution 2 (1×Taq buffer (SolGent), 0.2 mM dNTP, 0.2 μM primer (P2, P4, or P6), 5 mM MgCl₂, 1×SYBR, and 0.05 U Taq polymerase (SolGent)) was prepared. 18 μL of the prepared real-time PCR mixture solution was mixed with 2 μL of each eluted detection aptamer in a real-time PCR tube. Samples were analyzed using an Applied Biosystems 7500 real-time PCR system. The real-time PCR proceeded by conducting a first process of maintaining at 96° C. for 15 seconds, at 55° C. for 10 seconds, and at 70° C. for 30 minutes once and then repeating a second process of maintaining at 96° C. for 15 seconds and at 70° C. for 1 minute 40 times.

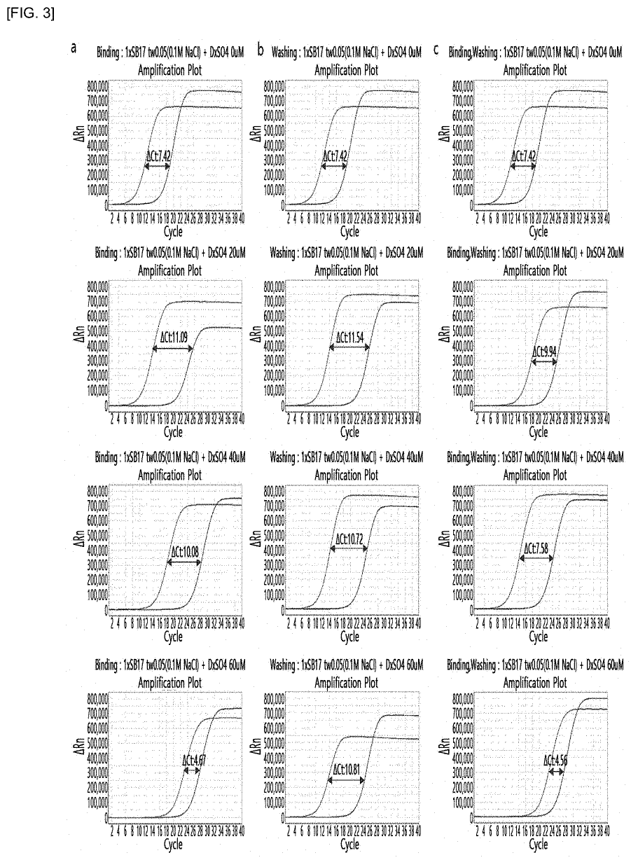

To examine effects of DₓSO₄ according to concentration in the binding step, eight samples of the target and non-target molecules were prepared using different concentrations (0 μM, 20 μM, 40 μM, and 60 μM) of DₓSO₄ as described above, and results as shown in FIG. 3a were obtained.

FIG. 3a shows real-time PCR results of VEGFR2 according to concentration of DₓSO₄ in the binding buffer. It was confirmed that the curve moves further to the right as the concentration of DₓSO₄ increases. Since the amounts of the protein and the aptamer are the same, a wider gap between two curves indicates a higher sensitivity to the target molecule. Upon comparison of the graphs showing results of different concentrations, it may be confirmed that the use of 20 μM or 40 μM showing a wider gap is suitable. It was confirmed that results obtained using another target molecule also showed the same pattern as shown in FIG. 3a.

### Experimental Example 2-2: Test with Different Concentrations of DₓSO₄ in Washing Step

A test was carried out in the same manner as in Experimental Example 2-1 until the washing process after blocking the magnetic beads. The magnetic beads prepared as described above were reacted with 20 pmol of a capture aptamer (SEQ ID NO: A1, A3, or A5) conjugated with biotin at the 5′-end thereof. The reaction was conducted in 100 μL of Binding Buffer 3 (1×SB17 and 0.05% tween20) in total at 600 rpm at room temperature for 15 minutes. In order to remove the capture aptamer that was not immobilized, the resultant was washed three times with 100 μL of Washing Buffer 3 (1×SB17, 0.05% tween20, and 0 μM or 20 μM or 40 μM or 60 μM DₓSO₄).

2 pmol of a target molecule or non-target molecule (IR, ErbB2, or VEGFR2) was added to the magnetic beads prepared as described above, followed by reaction in 100 μL of Binding Buffer 3 in total at 600 rpm at room temperature for 10 minutes. The target molecule not bound to the capture aptamer was washed out three times with 100 μL of Washing Buffer 3.

2 pmol of a detection aptamer (SEQ ID NO: A2, A4, or A6) was added to the magnetic beads prepared as described above, followed by reaction in 100 μL of Binding Buffer 3 in total at 600 rpm at room temperature for 10 minutes. Unreacted detection aptamer was washed out three times with 100 μL of Washing Buffer 3. 30 μL of 2 mM NaOH was added thereto, followed by reaction at 600 rpm at room temperature for 10 minutes to elute the detection aptamer.

Real-time PCR mixture solution 2 was prepared. 18 μL of the prepared real-time PCR mixture solution was mixed with 2 μL of each eluted detection aptamer in a real-time PCR tube. Samples were analyzed using an Applied Biosystems 7500 real-time PCR system. The real-time PCR proceeded by conducting a first process of maintaining at 96° C. for 15 seconds, at 55° C. for 10 seconds, and at 70° C. for 30 minutes once and then repeating a second process of maintaining at 96° C. for 15 seconds and at 70° C. for 1 minute 40 times.

To examine effects of DₓSO₄ according to concentration in the washing step, eight samples of the target and non-target molecules were prepared using different concentrations (0 μM, 20 μM, 40 μM, and 60 μM) of DₓSO₄ as described above, and results as shown in FIG. 3b were obtained.

FIG. 3b shows real-time PCR results of VEGFR2 according to concentration of DₓSO₄ in the washing buffer. It was confirmed that the curve moves further to the right as the concentration of DₓSO₄ increases in the same manner as in the case of the binding buffer. Upon comparison of the graphs showing results of different concentrations, it may be confirmed that the use of 20 μM or 40 μM showing a wider gap is suitable. It was confirmed that results obtained using another target molecule also showed the same pattern as shown in FIG. 3b.

### Experimental Example 2-3: Test with Different Concentrations of DₓSO₄ in Binding and Washing Step

A test was carried out in the same manner as in Experimental Example 1 until the washing process after blocking the magnetic beads. The magnetic beads prepared as described above were reacted with 20 pmol of a capture aptamer (SEQ ID NO: A1, A3, or A5) conjugated with biotin at the 5′-end thereof. The reaction was conducted in 100 μL of Binding Buffer 2 in total at 600 rpm at room temperature for 15 minutes. In order to remove the capture aptamer that was not immobilized, the resultant was washed three times with 100 μL of Washing Buffer 3.

2 pmol of a target molecule or non-target molecule (IR, ErbB2, or VEGFR2) was added to the magnetic beads prepared as described above, followed by reaction in 100 μL of Binding Buffer 2 in total at 600 rpm at room temperature for 10 minutes. The target molecule not bound to the capture aptamer was washed out three times with 100 μL of Washing Buffer 3.

2 pmol of a detection aptamer (SEQ ID NO: A2, A4, or A6) was added to the magnetic beads prepared as described above, followed by reaction in 100 μL of Binding Buffer 2 in total at 600 rpm at room temperature for 10 minutes. Unreacted detection aptamer was washed out three times with 100 μL of Washing Buffer 3. 30 μL of 2 mM NaOH was added thereto, followed by reaction at 600 rpm at room temperature for 10 minutes to elute the detection aptamer.

Real-time PCR mixture solution 2 was prepared. 18 μL of the prepared real-time PCR mixture solution was mixed with 2 μL of each eluted detection aptamer in a real-time PCR tube. Samples were analyzed using an Applied Biosystems 7500 real-time PCR system. The real-time PCR proceeded by conducting a first process of maintaining at 96° C. for 15 seconds, at 55° C. for 10 seconds, and at 70° C. for 30 minutes once and then repeating a second process of maintaining at 96° C. for 15 seconds and at 70° C. for 1 minute 40 times.

To examine effects of DₓSO₄ according to concentration in the binding and washing steps, eight samples of the target and non-target molecules were prepared according to the concentration (0 μM, 20 μM, 40 μM, and 60 μM) of DₓSO₄ as described above, and results as shown in FIG. 3c were obtained.

FIG. 3c shows real-time PCR results of VEGFR2 according to concentration of DₓSO₄ in the washing buffer. It was confirmed that the curve moves further to the right as the concentration of DₓSO₄ increases in the same manner as in the cases of Experimental Examples 2-1 and 2-2. Upon comparison of the graphs showing results of different concentrations, it may be confirmed that the use of 20 μM showing a wider gap is suitable, but the effects thereof were lower than those of FIGS. 3a and 3b. It was confirmed that results obtained using another target molecule also showed the same pattern as shown in FIG. 3c.

### Experimental Example 3: Single Diagnosis Using Aptamer Pair

An experiment was carried out in the same manner as in Experimental Example 2-1 until the washing process after blocking the magnetic beads. The magnetic beads prepared as described above were reacted with 20 pmol of a capture aptamer (SEQ ID NO: A1, A3, or A5) conjugated with biotin at the 5′-end thereof. The reaction was conducted in 100 μL of Binding Buffer 1 in total at 600 rpm at room temperature for 15 minutes. In order to remove the capture aptamer that was not immobilized, the resultant was washed three times with 100 μL of Washing Buffer 4 (1×SB17, 0.05% tween20, and 20 μM DₓSO₄).

2 pmol of a target molecule or non-target molecule (IR, ErbB2, or VEGFR2) was added to the magnetic beads prepared as described above, followed by reaction in 100 μL of Binding Buffer 1 in total at 600 rpm at room temperature for 10 minutes. The target molecule not bound to the capture aptamer was washed out three times with 100 μL of Washing Buffer 4.

2 pmol of a detection aptamer (SEQ ID NO: A2, A4, or A6) was added to the magnetic beads prepared as described above, followed by reaction in 100 μL of Binding Buffer 1 in total at 600 rpm at room temperature for 10 minutes. Unreacted detection aptamer was washed out three times with 100 μL of Washing Buffer 4. 30 μL of 2 mM NaOH was added thereto, followed by reaction at 600 rpm at room temperature for 10 minutes to elute the detection aptamer.

Real-time PCR mixture solution 2 was prepared. 18 μL of the prepared real-time PCR mixture solution was mixed with 2 μL of each eluted detection aptamer in a real-time PCR tube. Samples were analyzed using an Applied Biosystems 7500 real-time PCR system. The real-time PCR proceeded by conducting a first process of maintaining at 96° C. for 15 seconds, at 55° C. for 10 seconds, and at 70° C. for 30 minutes once and then repeating a second process of maintaining at 96° C. for 15 seconds and at 70° C. for 1 minute 40 times.

Nine samples of the target and non-target molecules were prepared and results as shown in FIG. 4 were obtained.

It was confirmed a curve of the target molecule was observed at a front portion and a curve of the non-target molecule was observed at a back portion based on the results under the conditions of Experimental Examples 1 and 2.

### Experimental Example 4: Comparison Between Single and Multiple Diagnosis Using Aptamer Pair

It was confirmed that single diagnosis was possible based on Experimental Example 3, samples were prepared as follows to identify whether the same result was obtained in multiple diagnosis.

An experiment was carried out in the same manner as in Experimental Example 2-1 until the washing process after blocking the magnetic beads. The magnetic beads prepared as described above were reacted respectively with 20 pmol of three types of capture aptamers (SEQ ID NO: A1, A3, and A5) conjugated with biotin at the 5′-end thereof. The reaction was conducted in 100 μL of Binding Buffer 1 in total at 600 rpm at room temperature for 15 minutes. In order to remove the capture aptamer that was not immobilized, the resultant was washed three times with 100 μL of Washing Buffer 4.

2 pmol of each of the proteins (IR, ErbB2, and VEGFR2) was added thereto to prepare 100 μL of Protein-binding Buffer 1. 100 μL of the protein-containing binding buffer was added to the magnetic beads prepared as described above, followed by reaction at 600 rpm at room temperature for 10 minutes. The target molecule not bound to the capture aptamer was washed out three times with 100 μL of Washing Buffer 4.

2 pmol of each of the three types of detection aptamers (SEQ ID NO: A2, A4, and A6) was added thereto to prepare 100 μL of Detection-binding Buffer 1. 100 μL of the detection-binding buffer was added to the magnetic beads prepared as described above, followed by reaction at 600 rpm at room temperature for 10 minutes. Unreacted detection aptamer was washed out three times with 100 μL of Washing Buffer 4. 30 μL of 2 mM NaOH was added thereto, followed by reaction at 600 rpm at room temperature for 10 minutes to elute the detection aptamer.

Real-time PCR mixture solution 2 was prepared. 18 μL of the prepared real-time PCR mixture solution was mixed with 2 μL of each eluted detection aptamer in a real-time PCR tube. The detection aptamer eluted from each sample was analyzed in three tubes using primers therefor. Samples were analyzed using an Applied Biosystems 7500 real-time PCR system. The real-time PCR proceeded by conducting a first process of maintaining at 96° C. for 15 seconds, at 55° C. for 10 seconds, and at 70° C. for 30 minutes once and then repeating a second process of maintaining at 96° C. for 15 seconds and at 70° C. for 1 minute 40 times.

Four samples were prepared using the target molecules in the same manner as in Experimental Example 3 and results as shown in FIG. 5 were obtained

As a result of comparing the conditions in multiple diagnosis and single diagnosis, results thereof were identical, and thus it was confirmed that the results of the multiple diagnosis developed by this research were consistent with the results of the single diagnosis.

### Experimental Example 5: Performance Test of Simultaneous Multiple Diagnosis Using Aptamer Pair

Performance of the multiple diagnosis was examined using the target molecule detectable based on the results obtained in the multiple diagnosis of Experimental Example 4.

An experiment was carried out in the same manner as in Experimental Example 4 until the process of immobilizing the capture aptamer. Each of the three types of proteins (IR, ErbB2, and VEGFR2) was added in an amount of 5 fmol to 2 pmol to prepare 100 μL of Protein-binding Buffer 1 in total. 100 μL of the protein-containing binding buffer was added to the magnetic beads prepared as described above, followed by reaction at 600 rpm at room temperature for 10 minutes. The target molecule not bound to the capture aptamer was washed out three times with 100 μL of Washing Buffer 4.

2 pmol of each of the three types of detection aptamers (SEQ ID NO: A2, A4, and A6) was added thereto to prepare 100 μL of Detection-binding Buffer 1. 100 μL of the detection-binding buffer was added to the magnetic beads prepared as described above, followed by reaction at 600 rpm at room temperature for 10 minutes. Unreacted detection aptamer was washed out three times with 100 μL of Washing Buffer 4. 30 μL of 2 mM NaOH was added thereto, followed by reaction at 600 rpm at room temperature for 10 minutes to elute the detection aptamer.

Real-time PCR mixture solution 2 was prepared. 18 μL of the prepared real-time PCR mixture solution was mixed with 2 μL of each eluted detection aptamer in a real-time PCR tube. The detection aptamer eluted from each sample was analyzed in three tubes using primers therefor. Samples were analyzed using an Applied Biosystems 7500 real-time PCR system. The real-time PCR proceeded by conducting a first process of maintaining at 96° C. for 15 seconds, at 55° C. for 10 seconds, and at 70° C. for 30 minutes once and then repeating a second process of maintaining at 96° C. for 15 seconds and at 70° C. for 1 minute 40 times.

FIG. 6 shows results of testing performance of a simultaneous multiple diagnosis system. It was confirmed that multiple diagnosis was possible even when the amount of the target molecule decreased to 5 fmol, and the same test results were observed in each of the target molecules.

### Experimental Example 6: Single Diagnosis Using Single Aptamer

In a different manner from that of Experimental Example 5, a diagnostic method using a single aptamer was performed.

To immobilize each target molecule, protein was diluted using a carbonate-bicarbonate solution (0.05 M, pH 9.6). 100 μL of each of the diluted protein and non-target molecule was added to each well of an ELISA-plate, followed by reaction at 4° C., at 600 rpm, for 12 hours. Unreacted protein was washed out once with 100 μL of Washing Buffer 1 at room temperature for 2 minutes at 600 rpm.

200 μL of Blocking Buffer 3 (3% BSA) was added to the plate, followed by blocking at room temperature for 1 hour at 600 rpm. The resultant was washed twice with 100 μL of Washing Buffer 1 at room temperature for 2 minutes at 600 rpm.

A detection aptamer (A2, A4, or A6) was added to each well of the plate, followed by reaction at room temperature at 800 rpm for 1 hour. The resultant was washed three times with 100 μL of Washing Buffer 1 for 2 minutes at 600 rpm. 30 μL of 2 mM NaOH was added thereto, followed by reaction at 600 rpm at room temperature for 10 minutes to elute a detection aptamer.

Real-time PCR mixture solution 2 was prepared. 18 μL of the prepared real-time PCR mixture solution was mixed with 2 μL of each eluted detection aptamer in a real-time PCR tube. Samples were analyzed using an Applied Biosystems 7500 real-time PCR system. The real-time PCR proceeded by conducting a first process of maintaining at 96° C. for 15 seconds, at 55° C. for 10 seconds, and at 70° C. for 30 minutes once and then repeating a second process of maintaining at 96° C. for 15 seconds and at 70° C. for 1 minute 40 times.

Nine samples of target and non-target molecules were prepared as described above, and results as shown in FIG. 7 were obtained.

It was confirmed a curve of the target molecule was observed at a front portion and a curve of the non-target molecule was observed at a back portion indicating that the aptamer was bound to the target molecule.

### Experimental Example 7: Simultaneous Multiple Diagnosis Using Single Aptamer

Based on Experimental Example 6, it was confirmed that the single diagnosis was possible and samples were prepared as follows to identify whether the same results were obtained by multiple diagnosis.

To immobilize the target molecule, protein was diluted using a carbonate-bicarbonate solution (0.05 M, pH 9.6). 100 μL of a mixed protein was added to each well of an ELISA-plate, followed by reaction at 4° C., at 600 rpm, for 12 hours. Unreacted protein was washed out once with 100 μL of Washing Buffer 1 at room temperature for 2 minutes at 600 rpm.

200 μL of Blocking Buffer 3 (3% BSA) was added to the plate, followed by blocking at room temperature for 1 hour at 600 rpm. The resultant was washed twice with 100 μL of Washing Buffer 1 at room temperature for 2 minutes at 600 rpm.

A detection aptamer (A2, A4, or A6) was added to each well of the plate, followed by reaction at room temperature at 800 rpm for 1 hour. The resultant was washed three times with 100 μL of Washing Buffer 1 for 2 minutes at 600 rpm. 30 μL of 2 mM NaOH was added thereto, followed by reaction at 600 rpm at room temperature for 10 minutes to elute a detection aptamer.

Real-time PCR mixture solution 2 was prepared. 18 μL of the prepared real-time PCR mixture solution was mixed with 2 μL of each eluted detection aptamer in a real-time PCR tube. Samples were analyzed using an Applied Biosystems 7500 real-time PCR system. The real-time PCR proceeded by conducting a first process of maintaining at 96° C. for 15 seconds, at 55° C. for 10 seconds, and at 70° C. for 30 minutes once and then repeating a second process of maintaining at 96° C. for 15 seconds and at 70° C. for 1 minute 40 times.

Four samples of the target molecule used in Experimental Example 6 were prepared as described above, and results as shown in FIG. 8 were obtained. Upon comparison of conditions between multiple diagnosis and single diagnosis, similar amplification curves were obtained, and thus it was confirmed that the results of the multiple diagnosis developed by this research were almost the same as those of the single diagnosis.

### Experimental Example 8: Performance Test of Simultaneous Multiple Diagnosis System Using Single Aptamer

Performance of the multiple diagnosis was verified using a target molecule detectable based on the results of the multiple diagnosis of Experimental Example 7.

To immobilize the target molecule, protein was diluted to 1 pmol to 5 fmol using a carbonate-bicarbonate solution (0.05 M, pH 9.6). 100 μL of a mixed protein was added to each well of an ELISA-plate, followed by reaction at 4° C., at 600 rpm, for 12 hours. Unreacted protein was washed out once with 100 μL of Washing Buffer 1 at room temperature for 2 minutes at 600 rpm.

200 μL of Blocking Buffer 3 (3% BSA) was added to the plate, followed by blocking at room temperature for 1 hour at 600 rpm. The resultant was washed twice with 100 μL of Washing Buffer 1 at room temperature for 2 minutes at 600 rpm.

A detection aptamer mixture (A2, A4 and A6) was added to each well of the plate, followed by reaction at room temperature at 800 rpm for 1 hour. The resultant was washed three times with 100 μL of Washing Buffer 1 for 2 minutes at 600 rpm. 30 μL of 2 mM NaOH was added thereto, followed by reaction at 600 rpm at room temperature for 10 minutes to elute a detection aptamer.

Real-time PCR mixture solution 2 was prepared. 18 μL of the prepared real-time PCR mixture solution was mixed with 2 μL of each eluted detection aptamer in a real-time PCR tube. Samples were analyzed using an Applied Biosystems 7500 real-time PCR system. The real-time PCR proceeded by conducting a first process of maintaining at 96° C. for 15 seconds, at 55° C. for 10 seconds, and at 70° C. for 30 minutes once and then repeating a second process of maintaining at 96° C. for 15 seconds and at 70° C. for 1 minute 40 times.

FIG. 9 shows results of a performance test of a simultaneous multiple diagnosis system. It was confirmed that multiple diagnosis was possible even when the amount of the target molecule decreased to 5 fmol, and the same test results were observed in each of the target molecules.

The aptamers and primer sequences used in the present invention are as shown in Tables 1 and 2 below.

TABLE 1
Information on Aptamer Sequence

| SEQ | | | Size |
|---|---|---|---|
| ID NO: | Sequence # | Sequence (5′-3′) | (bp) |
| 1 | Biotin-1652-49 | 5′-GCC TGN AAG GNN NAA GCN NGG CCN AAN | 42 |
| | | GGN GCN ANC AGG CNC-3′ | |
| 2 | OH-1652-20 | 5′-GAG TGA CCG TCT GCC TGN NAN CCA CNA | 74 |
| | | NGG CNN CNC ANN CAA ANA AGN GCG ANC GAN | |
| | | CAG CCA CAC CAC CAG CC-3′ | |
| 3 | Biotin-1194-35 | 5′-VCC VGG CAV GVV CGA VGG AGG CCV VVG | 40 |
| | | AVV ACA GCC CAG A-3′ | |
| 4 | OH-1194-34-01 | 5′-GAG TGA CCG TCT GCC TGA VGV VAG AGV | 74 |
| | | VVG CCV GAG VGC CVC GCA AGG GCG VAA CAA | |
| | | CAG CCA CAC CAC CAG CC-3′ | |
| 5 | biotin-2041-19-06 | 5′-TGA CGA GCN ACG ACG NCN GGN GNA ANN | 57 |
| | | NAN AAA GAC ACN GNG NAN ANC AAC AAC | |
| | | AGA-3′ | |
| 6 | OH-2041-06-01 | 5′-GAT GTG AGT GTG TGA CGA GCC NGA NAN | 80 |
| | | NCN GCG NAN NAG CCC NAN NAA NGN NAC GGN | |
| | | AGC AAC AAC AGA ACA AGG AAA GG-3′ | |

*N: BzdU, V: NapdU (using modified nucleic acid)

TABLE 2
Information on Primer Sequence

| | | (5′-3′) | Length |
|---|---|---|---|
| P1 | forward | CTG TAA GGT TTA AGC TTG GCC TAA TG | 26 |
| | reverse | GGA CGA GCA GAG CCT GAT AGC | 21 |
| P2 | forward | GAG TGA CCG TCT GCC TGT TAT CCA C | 25 |
| | reverse | GGC TGG TGG TGT GGC TGA TCG ATC | 24 |
| P3 | forward | CTG GCA TGT TCG ATG GAG GCC | 21 |
| | reverse | GGA CGA GCA TCT GGG CTG TAA TC | 23 |
| P4 | forward | GAG TGA CCG TCT GCC TGA TGT TAG | 24 |
| | reverse | GGC TGG TGG TGT GGC TGT TGT TAC | 24 |
| P5 | forward | GGA CGA GCACTA CGA CGT CTG | 21 |
| | reverse | GGA CGA GCA CAC AGT GTC TTT ATA AA | 26 |
| P6 | forward | GGA CGA GCA CGA GTA AAT GAA TG | 23 |
| | reverse | GGA CGA GCA GAA ATG CTC AAA C | 22 |

The above description of the present invention is provided for the purpose of illustration, and it would be understood by those skilled in the art that various changes and modifications may be made without changing the technical conception and essential features of the present invention. Thus, it is clear that the above-described embodiments are illustrative in all aspects and do not limit the present invention. The various embodiments disclosed herein are not intended to be limiting, with the true scope and spirit being indicated by the following claims. The present invention is to be limited only by the terms of the appended claims, along with the full scope of equivalents to which such claims are entitled.

1: A method of detecting a target molecule, the method comprising:
(i) bringing an aptamer recognizing a target region of a target molecule into contact with the target molecule; and
(ii) performing a polymerase chain reaction (PCR) using, as a template, an aptamer forming a complex via the contact and a bound aptamer in a complex of the target molecule.

2: The method of claim 1, wherein multiplex-PCR is performed using one or more types of target molecules.

3: The method of claim 1, wherein the multiplex-PCR is performed by:
(i) simultaneously detecting and quantifying one or more target molecules by simultaneously reacting the target molecules with an aptamer in a well or tube; or
(ii) simultaneously detecting and quantifying one or more target molecules by reacting the target molecules with an aptamer in one or more wells or tubes.

4: The method of claim 1, wherein the polymerase chain reaction (PCR) is real-time PCR.

5: The method of claim 4, wherein the real-time PCR is a TaqMan probe PCR using an intercalating fluorescent dye.

6: The method of claim 1, wherein the aptamer of the step (i) is an aptamer pair comprising a capture aptamer recognizing a target region and a detection aptamer recognizing a target region and used as a template, or a single aptamer recognizing a target region and used as a template.

7: The method of claim 6, wherein the aptamer pair or the single aptamer is present in types corresponding to types of target molecules or target regions.

8: The method of claim 6, wherein the aptamer is a modified nucleic acid or non-modified nucleic acid.

9: The method of claim 1, wherein the target molecule is present in an isolated sample and the target molecule contained in the sample is detected by the method of detecting a target molecule.

10: The method of claim 1, wherein the target molecule comprises at least one selected from the group consisting of a protein, a peptide, a carbohydrate, a polysaccharide, a glycoprotein, a hormone, a receptor, an antigen, an antibody, a virus, a cofactor, a drug, a dye, a growth factor, and a controlled substance.

11: The method of claim 9, wherein the sample comprises at least one selected from the group consisting of a biological sample, an environmental sample, a chemical sample, a pharmaceutical sample, a food sample, an agricultural sample, and a livestock sample.

12: The method of claim 11, wherein the sample comprises at least one selected from the group consisting of whole blood, leukocytes, peripheral blood mononuclear cells, plasma, serum, sputum, breath, urine, semen, saliva, meningeal fluid, amniotic fluid, glandular fluid, lymph fluid, nipple aspirate, bronchial aspirate, synovial fluid, joint aspirate, cells, cell extract, stool, tissue extract, biopsy tissue, and cerebrospinal fluid.

13: The method of claim 1, wherein the performing of PCR is conducted by detecting and quantifying the target molecule.

14: The method of claim 6, wherein when the aptamer pair is used, the method comprises the following steps:
(a) forming a first complex by binding a first capture aptamer to a solid support;
(b) binding a target molecule to the first complex of the step (a);
(c) forming a second complex by binding a second capture aptamer to the target molecule of the step (b); and
(d) isolating the second capture aptamer from the second complex of the step (c) and performing a polymerase chain reaction (PCR).

15: The method of claim 6, wherein one or more types of target molecules are detected using an aptamer recognizing one or more types of particularly antigens including a complex in which the target molecule is bound to the first complex before the step (c);

16: The method of claim 6, wherein when the single aptamer is used, the method comprises the following steps:
(a) binding a target molecule to a solid support;
(b) forming a complex by binding a capture aptamer to the target molecule of the step (a); and
(c) isolating the capture aptamer from the complex of the step (b) and performing a polymerase chain reaction (PCR).

17: The method of claim 14 or 16, further comprising selectively removing the capture aptamer not bound to the target molecule in the complex or not bound to the complex.

18: The method of claim 14, further comprising blocking the solid support using a blocking buffer to prevent non-specific binding thereof before binding the aptamer to the solid support in the step (a).

19: The method of claim 18, wherein the blocking buffer comprises at least one selected from the group consisting of bovine serum albumin (BSA), salmon sperm DNA, herring sperm DNA, skim milk, and casein.

20: The method of claim 14, wherein the solid support comprises at least one selected from the group consisting of a magnetic bead, a polymer bead, an agarose bead, a polystyrene bead, an acrylamide bead, a solid core bead, a porous bead, a paramagnetic bead, a glass bead, a controlled pore bead, a microtiter well, a cycloolefin copolymer substrate, a membrane, a plastic substrate, nylon, a Langmuir-Blodgett film, glass, a germanium substrate, a silicon substrate, a silicon wafer chip, a flow through chip, a microbead, a polytetrafluoroethylene substrate, a polystyrene substrate, a gallium arsenide substrate, a gold substrate, and a silver substrate.

21: The method of claim 1, wherein the aptamer is a single-stranded nucleic acid or a double-stranded nucleic acid.

22: The method of claim 1, wherein the aptamer is DNA, RNA, or a combination thereof.

23: A composition for detecting a target molecule comprising an aptamer recognizing a target region of a target molecule, wherein the aptamer is used as a template for polymerase chain reaction (PCR).

24: The composition of claim 23, wherein multiplex-PCR is performed using one or more types of target molecules.

25: The composition of claim 23, wherein the polymerase chain reaction (PCR) is real-time PCR.

26: The composition of claim 23, wherein the aptamer is an aptamer pair comprising a capture aptamer recognizing a target region and a detection aptamer recognizing a target region and used as a template, or a single aptamer recognizing a target region and used as a template.
